(19) United States
(12) Patent Application Publication
(10) Pub. No.: US 2017/0136138 A1
(43) Pub. Date: May 18, 2017

(54) Aroma Burner

(71) Applicant: Xiaoyuan Ma, Shenzhen (CN)

(72) Inventor: Xiaoyuan Ma, Shenzhen (CN)

(21) Appl. No.: 14/986,672

(22) Filed: Jan. 2, 2016

(30) Foreign Application Priority Data
Nov. 18, 2015 (CN) 201520920260.0

(51) Int. Cl. A61L 9/03

(52) U.S. Cl. CPC A61L 9/03

(57) ABSTRACT

The present invention relates to an aroma burner comprising a burner body and a base; the upper end in the burner body is provided with a ring slot internally provided with microcrystal glass panels; the microcrystal glass panels are affixed on the edge of the slot of the burner body through silica gel; a wax storage bowl is disposed on the upper ends of the microcrystal glass panels; a plurality of aroma wax blocks are placed in the wax storage bowl; the lower ends of the microcrystal glass panels are applied with a silver paste coating; the base is provided with a power socket; the base is also provided with a transparent plastic box; the plastic box is provided with a micro-control circuit board; the circuit board is connected with the silver paste coating and the power switch.

### BACKGROUND OF THE INVENTION

Technical Field

The present invention relates to the technical field of burners, in particular to an aroma burner.

Description of Related Art

Aroma burners can be used on many occasions in daily life. Aroma burners can improve the environmental health, remove peculiar odors, purify air, refresh people, keep the mind clear, and enhance work efficiency. Aromas can also pacify intense emotion, release pressure, and improve symptoms such as sleeplessness and dizziness. Aromas can also create a romantic atmosphere, make people feel warm and happy, and stimulate the potential immune competence of people. At present, conventional aroma burners all heat aroma wax with lamps, resulting in an undesirable heating effect and poor safety.

### BRIEF SUMMARY OF THE INVENTION

The objective of the present invention aims to provide an aroma burner to solve the above mentioned problems.

To solve the above mentioned problems, the utility model adopts the following technical solution.

An aroma burner comprises a burner body and a base; the upper end in the burner body is provided with a ring slot; the slot is internally provided with microcrystal glass panels; the microcrystal glass panels are affixed on the edge of the slot of the burner body through silica gel; a wax storage bowl is disposed on the upper ends of the microcrystal glass panels; a plurality of aroma wax blocks are placed in the wax storage bowl; the base is provided with a power socket; the base is also provided with a transparent plastic box; the plastic box is internally provided with a micro-control circuit board; a power wire penetrates the power socket to be connected with the circuit board; the circuit board is connected with the silver paste coating via one end of the power wire, and the other end is connected with the power switch out of the power socket.

Preferably, the circuit board is provided with an LED lamp; the LED lamp is connected with the circuit board through one end of the power wire, while the other end is connected with the silver paste coating.

Preferably, the burner body is provided with a plurality of light holes.

Preferably, the wax storage bowl is made of ceramics, glass, or iron materials.

Preferably, the lower end of the burner body is provided with two or more support legs.

Preferably, the base is connected to the burner body through screw threads.

The utility model has the following beneficial effects:

The utility model comprises a burner body and a base; the upper end in the burner body is provided with a ring slot; the slot is internally provided with microcrystal glass panels; the microcrystal glass panels are affixed on the edge of the slot of the burner body through silica gel; a wax storage bowl is disposed on the upper ends of the microcrystal glass panels; a plurality of aroma wax blocks are placed in the wax storage bowl; the lower ends of the microcrystal glass panels are applied with a silver paste coating; the base is provided with a power socket; the base is also provided with a transparent plastic box; the plastic box is internally provided with a micro-control circuit board; a power wire penetrates the power socket to be connected with the circuit board; the circuit board is connected with a silver paste coating via one end of the power wire, and the other end is connected with the power switch out of the power socket. After the aroma burner is electrified, the silver paste coating directly carries out electric conduction and heating to melt the aroma blocks in the wax storage bowl, sending out a fresh smell. With the temperature rise, the effect is better.

In the utility model, the LED lamp is disposed on the base; the circuit board is disposed in the base; the LED lamp is connected with the circuit board via the power wire; one end of the power wire is connected with the circuit board, while other end is connected with the silver paste coating; and the burner body is provided with a plurality of light holes. When the aromatic smell spreads, the LED lamp is powered via the silver paste coating, and emits light out via the light holes to create a comfortable environment.

In the utility model, the lower end of the burner body is provided with two or more support legs. In this way, the burner body can be well fixed on a plane and used conveniently.

The utility model is simple in structure, easy to manufacture, and suitable for industrial production.

### BRIEF DESCRIPTION OF THE SEVERAL VIEWS OF THE DRAWINGS

FIG. 1 is a structural view of the utility model.

In the figure: 1. wax storage bowl; 2. aroma block; 3. microcrystal glass panel; 4. silver paste coating; 5. slot; 6. burner body; 7. LED lamp; 8. power wire; 9. power socket; 10. circuit board; 11. support leg; 12. light hole; 13. base; 14. power switch; 15. plastic box.

### DETAILED DESCRIPTION OF THE INVENTION

The utility model is further described with reference to the attached drawings and specific embodiments.

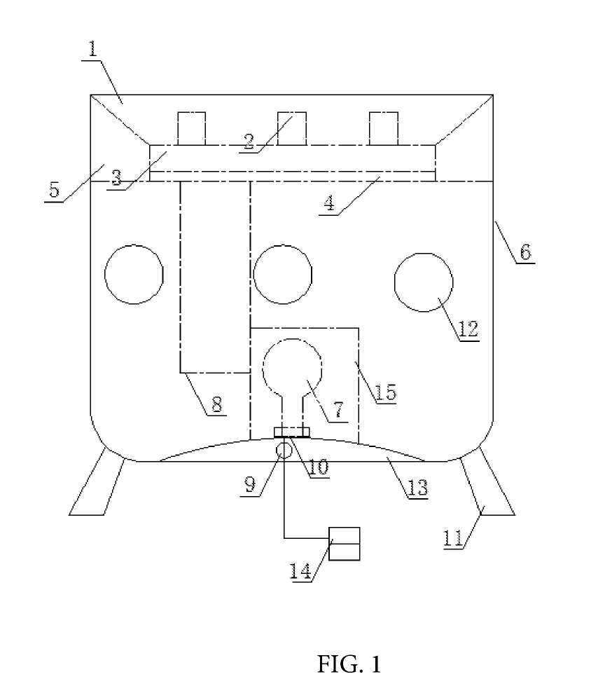

As shown in FIG. 1, the present invention can be an aroma burner, comprising a burner body 6 and a base 13; the upper end of the burner body 6 is provided with a ring slot 5; the ring slot 5 is internally provided with a plurality of microcrystal glass panels 3; the microcrystal glass panels 3 are affixed on the edge of the slot 5 through silica gel; a wax storage bowl 1 is disposed on the upper ends of the microcrystal glass panels 3; a plurality of aroma wax blocks 2 are placed in the wax storage bowl 1; the lower ends of the microcrystal glass panels 3 are applied with a silver paste coating 4; the base 13 is provided with a power socket 9; the base is also provided with a transparent plastic box 15; the plastic box 15 is provided with a micro-control circuit board 10; a power wire penetrates the power socket 9 to be connected with the circuit board 10; the circuit board 10 is connected to the silver paste coating 4 via the power wires 8 on one end, and connected with the power switch 14 out of the power hole on the other end. After the aroma burner is electrified, the silver paste coating 4 directly carries out electric conduction and heating to melt the aroma blocks 2 in the wax storage bowl 1, sending out a fresh fragrant. The effect of the burner increases as temperature increases.

The circuit board 10 is provided with an LED lamp 7; the LED lamp 7 is connected with the circuit board 10 through one end of the power wire, while the other end is connected with the silver paste coating 4. The burner body 6 is provided with a plurality of light holes 12. When the aromatic smell spreads, the LED lamp is powered via the silver paste coating 4, and emits light via the light holes 12 to create a comfortable environment. The wax storage bowl 1 can be made of ceramics, glass, or iron materials. The lower end of the burner body 6 is provided with two or more support legs 11 so the burner body 6 can be well fixed on a plane, and conveniently used. The base 13 is connected to the burner body 6 via screw threads, so it is very convenient to dismantle the LED lamp 7 from base 13.

Finally, it should be noted that the above embodiments are used to describe the utility model instead of limiting the technical solution of the invention, so although the description depicts the utility model with reference to the above embodiments in detail, those ordinarily skilled in this field shall understand that thee technical solution of the utility model has modifications or equivalent replacements, and those modifications or equivalent replacements within the spirit or scope of the utility model shall fall within the scope of the claims of the utility model.

What is claimed is:

1. An aroma burner, comprising:
a burner body and a base; wherein an upper end in the burner body is provided with a ring slot; wherein the slot is internally provided with microcrystal glass panels;
wherein the microcrystal glass panels are affixed on the edge of the slot of the burner body through silica gel; wherein a wax storage bowl is disposed on the upper ends of the microcrystal glass panels; wherein a plurality of aroma wax blocks are placed in the wax storage bowl; wherein the lower ends of the microcrystal glass panels are applied with a silver paste coating; wherein the base is provided with a power socket;
wherein the base is provided with a transparent plastic box; wherein the plastic box is provided with a micro-control circuit board; wherein a power wire penetrates the power socket to be connected with the circuit board; wherein the circuit board is connected with the silver paste coating via one end of the power wire, and the other end of the power wire is connected with a power switch.

2. The aroma burner according to claim 1, wherein said circuit board is provided with an LED lamp; wherein the LED lamp is connected with the circuit board on one end and connected with the silver paste coating on the other end through power wire.

3. The aroma burner according to claim 1, wherein said burner body is provided with a plurality of light holes.

4. The aroma burner according to claim 1, wherein said wax storage bowl is made of ceramics, glass or iron materials.

5. The aroma burner according to claim 1, wherein lower end of said burner body is provided with two or more support legs.

6. The aroma burner according to claim 1, wherein said base is connected to the burner body via screw threads.
